(19) United States
(12) Patent Application Publication
(10) Pub. No.: US 2014/0274984 A1
(43) Pub. Date: Sep. 18, 2014

(54) ACID ADDITION SALTS OF 5ALPHA-HYDROXY-6BETA-[2-(1H-IMIDAZOL-4-YL)ETHYLAMINO]CHOLESTAN-3BETA-OL

(71) Applicants: INSERM (INSTITUT NATIONAL DE LA SANTÉ ET DE LA RECHERE MÉDICALE, Paris (FR); AFFICHEM, Toulouse (FR)

(72) Inventors: Marc Poirot, Toulouse (FR); Sandrine Poirot, Toulouse (FR); Philippe de Medina, Toulouse (FR); Michael Paillasse, Toulouse (FR)

(73) Assignees: AFFICHEM, Toulouse (FR); INSERM (Institute National de la Sante et de la Recherche Medicale, Paris (FR)

(21) Appl. No.: 14/352,160

(22) PCT Filed: Oct. 17, 2012
(86) PCT No.: PCT/EP2012/070588

§ 371 Date: Apr. 16, 2014

(30) Foreign Application Priority Data
Oct. 18, 2011 (EP) 11306346.5

(51) Int. Cl. C07C 309/30; C07C 59/255; C07C 55/10; C07C 63/08; C07C 309/29; C07C 65/11; C07C 55/12; C07C 55/08; C07C 57/15; C07C 309/04; C07C 53/10; C07C 59/265; C07C 59/08; C07C 59/245; C07J 43/00

(52) U.S. Cl. CPC C07C 309/30; C07J 43/003; C07C 59/255; C07C 55/10; C07C 63/08; C07C 309/29; C07C 65/11; C07C 55/12; C07C 55/08; C07C 57/15; C07C 309/04; C07C 53/10; C07C 59/265; C07C 59/08; C07C 59/245
USPC 514/176; 540/106

(57) ABSTRACT

The present invention relates to acid addition salts of 5α-hydroxy-6β-[2-(1H-imidazol-4-yl)ethylamino]cholestan-3β-ol, to their preparation and to applications thereof.

The present invention relates to acid addition salts of 5α-hydroxy-6β-[2-(1H-imidazol-4-yl)ethylamino]cholestan-3β-ol, to their preparation and to applications thereof.

The pharmaceutically active compound 5α-hydroxy-6β-[2-(1H-imidazol-4-yl)ethylamino]cholestan-3β-ol is known under the name Dendrogenin A. Its structural formula is the following:

[chemical structure]

Dendrogenin A is disclosed in WO0389449 and de Medina et al (J. Med. Chem., 2009) as free base. The solubility in water of the free base is 0.47 mg/ml.

However, acid addition salts of Dendrogenin A have never been disclosed up to now.

The present invention is directed to acid addition salts of Dendrogenin A which possess a remarkable solubility in water (up to 130 times higher than the solubility of the free base).

Due to their remarkable solubility, the salts of the present invention are expected to be more bioavailable than the free base when they are injected, thereby allowing the administration of higher doses.

The salts of the present invention are acid addition salt of 5α-hydroxy-6β-[2-(1H-imidazol-4-yl)ethylamino]cholestan-3β-ol formed with an acid selected from the group consisting of:
An inorganic acid,
An acyclic aliphatic carboxylic or sulfonic acid comprising no more than 8 carbon atoms, and
An aromatic carboxylic or sulfonic acid comprising no more than 4 aryl group.

The salts of the invention can have a solubility in water comprised between 1 and 70 mg/ml, in particular between 1.4 and 65 mg/ml, more specifically:
between 35 and 65 mg/ml, such as salts formed with L-lactic acid, malonic acid, L-malic acid or tartric acid (D or L);
between 20 and 35 mg/ml, such as benzenesulfonic acid, benzoic acid, succinic acid, 4-methylbenzene sulfonic acid or hydrochloride acid;
between 10 and 20 mg/ml, such as sulphuric acid, fumaric acid, glutaric acid, or mesylic acid;
or between 1.4 and 10 mg/ml, such as citric acid, acetic acid or pamoic acid.

The salts of the invention can be prepared by the reaction of Dendrogenin A as free base with one of the above listed acids. The solvent used for the reaction may be for instance ethanol, water or toluene. It may be heated during the reaction. The resulting salt may be recovered according to methods well known by the one skilled in the art.

The acid addition salts of Dendrogenin A of the present invention may be formed from inorganic acids.

In one embodiment, the inorganic acids do not include hydrochloride acid.

Preferred inorganic acids are selected from the group consisting of hydrochloride acid and sulfuric acid.

The acid addition salts of Dendrogenin A formed with inorganic acids can have a solubility in water comprised between 15 and 25 mg/ml.

The acid addition salts of Dendrogenin A of the present invention may also be formed from acyclic aliphatic carboxylic acids comprising no more than 8 carbon atoms.

The acyclic aliphatic carboxylic acids preferably comprise between 2 and 6 carbon atoms, preferably between 3 and 4 carbon atoms.

They include monocarboxylic acids or dicarboxylic acids.

Mono carboxylic acids may be substituted with at least one hydroxyl group.

Preferred monocarboxylic acids include acetic acid or L-lactic acid (2(S)-hydroxypropanoic acid).

Dicarboxylic acids may also be substituted with at least one hydroxyl group.

Preferred dicarboxylic acids include tartaric acid, L-malic acid, succinic acid, malonic acid, fumaric acid and glutaric acid.

Mono or dicarboxylic acids substituted with at least one hydroxyl group preferably include L-tartaric acid, D-tartaric acid, L-malic acid, citric acid, or 2(S)-hydroxypropanoic acid.

Acyclic aliphatic carboxylic also include tricarboxylic acids such as citric acid or acids containing one unsaturation, such as malonic acid.

The acid addition salts of Dendrogenin A formed with acyclic aliphatic carboxylic can have a solubility in water comprised between 4 and 60 mg/ml.

Preferred acyclic aliphatic carboxylic acids are selected from the group consisting of L-lactic acid, malonic acid, L-malic acid, and tartric acid (D or L).

Acid addition salts of Dendrogenin A formed with such acids can have a solubility in water comprised between 35 and 65 mg/ml.

The acid addition salts of Dendrogenin A of the present invention may also be formed from acyclic aliphatic sulfonic acids comprising no more than 8 carbon atoms, such as mesylic acid.

The acid addition salts of Dendrogenin A of the present invention may also be formed from aromatic carboxylic or sulfonic acids comprising no more than 4 aryl group.

Aromatic carboxylic or sulfonic acids comprising no more than 4 aryl group preferably contain no more than one aryl group, such as benzenesulfonic acid, benzoic acid, or 4-methylbenzenesulfonic acid.

The acid addition salts of Dendrogenin A formed with such acids can have a solubility in water comprised between 20 and 35 mg/ml.

Preferred aromatic carboxylic acids containing up to 4 aryl groups include pamoic acid (4,4′methylenebis(3-hydroxy-2-naphtoic acid)).

The present invention also relates to a pharmaceutical composition comprising a pharmaceutically acceptable carrier and one of the above mentioned acid addition salts of Dendrogenin A.

The present invention also relates to one of the above mentioned acid addition salts of Dendrogenin A for use in the treatment of neurodegenerative diseases, cancers or for activating the immune system of a patient.

The following Examples illustrate the preparation of acid addition salts of Dendrogenin A according to the present invention.

### EXAMPLE 1

### 5alpha-Hydroxy-6beta-[2-(1H-imidazol-4-yl)ethylamino]cholestan-3beta-ol, hydrochloride

Aqueous hydrochloride acid (0.9 g, 37%) is added to a solution of 5alpha-Hydroxy-6beta-[2-(1H-imidazol-4-yl)ethylamino]cholestan-3beta-ol (5.14 g, 10 mmole) in ethanol (10 ml). The solution is evaporated to dryness under reduced pressure and the resulting residue is re-crystallized from methanol. The product is filtered off and re-crystallized from ethanol to afford, after filtering and drying, 5alpha-Hydroxy-6beta-[2-(1H-imidazol-4-yl)ethylamino]cholestan-3beta-ol, hydrochloride as a pale-yellow crystalline solid, having the following analytical properties: analysis found: C, 77.11; H, 10.40; N, 7.79; Cl, 6.58%. H2O, 1.75%. Calculated for C36H56ClN3O2-0.52H2O: C, 77.09; H, 10.37; N, 7.78; Cl, 6.57%. H2O, 1.73%.

### EXAMPLE 2

### 5alpha-Hydroxy-6beta-[2-(1H-imidazol-4-yl)ethylamino]cholestan-3beta-ol, L-tartrate

5 alpha-Hydroxy-6beta-[2-(1H-imidazol-4-yl)ethylamino]cholestan-3beta-ol (5.4 g, 10 mmol) is added to a solution of (2R,3R)2,3-dihydroxybutanedioic acid (L-(+)-tartaric acid; Fluka; 1.5 g 10 mmol) in ethanol (40 ml). The solution is evaporated to dryness under reduced pressure and the resulting residue is re-crystallized from methanol. The product is filtered off and re-crystallized from to afford, after filtering and drying, 5alpha-Hydroxy-6beta-[2-(1H-imidazol-4-yl)ethylamino]cholestan-3beta-ol, tartrate as a pale-yellow crystalline solid, having the following analytical properties: analysis found: C, 66.61; H, 9.42; N, 6.48%. H2O, 2.23%. Calculated for C36H61N3O8-0.8H2O: C, 66.60; H, 9.46; N, 6.48%. H2O, 2.22%.

### EXAMPLE 3

### 5alpha-Hydroxy-6beta-[2-(1H-imidazol-4-yl)ethylamino]cholestan-3beta-ol, L-malate

5alpha-Hydroxy-6beta-[2-(1H-imidazol-4-yl)ethylamino]cholestan-3beta-ol (5.4 g, 10 mmol) is added to a solution of (2S)-(−)-hydroxybutanoic acid (L-(−)-malic acid; Fluka; 1.34 g 10 mmol) in water/Ethanol (1:1) (40 ml). The solution is evaporated to dryness under reduced pressure and the resulting residue is re-crystallized from methanol. The product is filtered off and re-crystallized from to afford, after filtering and drying, 5alpha-Hydroxy-6beta-[2-(1H-imidazol-4-yl)ethylamino]cholestan-3beta-ol, malate as a pale-yellow crystalline solid, having the following analytical properties: analysis found: C, 67.22; H, 9.53; N, 6.51%. H2O, 0.65%. Calculated for C36H61N3O7-0.23H2O: C, 67.20; H, 9.49; N, 6.53%. H2O, 0.64%.

### EXAMPLE 4

### 5alpha-Hydroxy-6beta-[2-(1H-imidazol-4-yl)ethylamino]cholestan-3beta-ol, succinate

5alpha-Hydroxy-6beta-[2-(1H-imidazol-4-yl)ethylamino]cholestan-3beta-ol (5.4 g, 10 mmol) is added to a solution of butanedioic acid (succinic acid; Fluka; 1.18 g 10 mmol) in Ethanol (40 ml). The solution is heated to 90° C., treated with water (18 g) and filtered. Upon cooling the product crystallizes and is filtered, dried to afford 5alpha-Hydroxy-6beta-[2-(1H-imidazol-4-yl)ethylamino]cholestan-3beta-ol, succinate as a pale-yellow crystalline solid, having the following analytical properties: analysis found: C, 68.99; H, 9.81; N, 6.72%. H2O, 0.78%. Calculated for C36H61N3O6-0.25H2O: C, 68.95; H, 9.74; N, 6.70%. H2O, 0.72%.

### EXAMPLE 5

### 5alpha-Hydroxy-6beta-[2-(1H-imidazol-4-yl)ethylamino]cholestan-3beta-ol, benzoate:

5alpha-Hydroxy-6beta-[2-(1H-imidazol-4-yl)ethylamino]cholestan-3beta-ol (5.4 g, 10 mmol) is added to a solution of benzoic acid (Fluka; 1.22 g 10 mmol) in toluene (40 ml). The solution is heated and filtered. Upon cooling the product crystallizes and is filtered, dried to afford 5alpha-Hydroxy-6beta-[2-(1H-imidazol-4-yl)ethylamino]cholestan-3beta-ol, benzoate as a pale-brown crystalline solid, having the following analytical properties: analysis found : C, 73.73; H, 9.66; N, 6.63%. Calculated for C39H61N3O4: C, 73.70; H, 9.61; N, 6.61%.

### EXAMPLE 6

### 5alpha-Hydroxy-6beta-[2-(1H-imidazol-4-yl)ethylamino]cholestan-3beta-ol, benzene sulfonate

5alpha-Hydroxy-6beta-[2-(1H-imidazol-4-yl)ethylamino]cholestan-3beta-ol (5.4 g, 10 mmol) is added to a solution of benzenesulfonic acid (Fluka; 1.61 g 10 mmol) in hot toluene (40 ml). The solution is evaporated to dryness under reduced pressure and the resulting residue is re-crystallized from ethanol-ethyl acetate. The product is filtered off and dried, to afford 5alpha-Hydroxy-6beta-[2-(1H-imidazol-4-yl)ethylamino]cholestan-3beta-ol, benzenesulfonate as a pale-yellow crystalline solid, having the following analytical properties: analysis found: C, 72.80; H, 9.89; N, 5.77%. H2O, 1.11%. Calculated for C38H61N3O5S-0.38H2O: C, 72.78; H, 9.85; N, 5.75%. H2O, 1.09%

### EXAMPLE 7

### 5alpha-Hydroxy-6beta-[2-(1H-imidazol-4-yl)ethylamino]cholestan-3beta-ol, pamoate

A mixture of 5alpha-Hydroxy-6beta-[2-(1H-imidazol-4-yl)ethylamino]cholestan-3beta-ol (5.4 g, 10 mmol) and 4,4′methylenebis(3-hydroxy-2-naphtoic acid) (Fluka; 3.88 g 10 mmol) is heated in ethanol (40 ml). Water is added (25 ml). Upon cooling the product crystallizes and is filtered, dried to afford 5alpha-Hydroxy-6beta-[2-(1H-imidazol-4-yl)ethylamino]cholestan-3beta-ol, pamoate as a pale-yellow crystalline solid, having the following analytical properties: analysis found: C, 74.96; H, 8.15; N, 4.78%. H2O, 2.37%. Calculated for C55H71N3O8-1.15H2O: C, 74.93; H, 8.08; N, 4.77%. H2O, 2.35%

### EXAMPLE 8

### 5alpha-Hydroxy-6beta-[2-(1H-imidazol-4-yl)ethylamino]cholestan-3beta-ol, glutarate

A solution of 1,5-pentanedioic acid (glutaric acid, Fluka, 660 mg, 5 mmole) is added to a solution of 5alpha-Hydroxy-6beta-[2-(1H-imidazol-4-yl)ethylamino]cholestan-3beta-ol (2.57 g, 5 mmole) in hot ethanol (100 ml). The solution is evaporated to dryness under reduced pressure and the resulting residue is re-crystallized from ethanol. The product is filtered off and re-crystallized from to afford, after filtering and drying, 5alpha-Hydroxy-6beta-[2-(1H-imidazol-4-yl)ethylamino]cholestan-3beta-ol, glutarate as a pale-yellow crystalline solid, having the following analytical properties: analysis found: C, 71.09; H, 10.74; N, 6.76%. H2O, 3.38%. Calculated for C37H63N3O6-1.15H2O: C, 71.07; H, 10.68; N, 6.72%. H2O, 3.31%

### EXAMPLE 9

### 5alpha-Hydroxy-6beta-[2-(1H-imidazol-4-yl)ethylamino]cholestan-3beta-ol, malonate:

A solution of 1,3-propanedioic acid (malonic acid, Fluka, 520 mg, 5 mmole) is added to a solution of 5alpha-Hydroxy-6beta-[2-(1H-imidazol-4-yl)ethylamino]cholestan-3beta-ol (2.57 g, 5 mmole) in hot ethanol (100 ml). The solution is evaporated to dryness under reduced pressure and the resulting residue is re-crystallized from ethanol. The product is filtered off and re-crystallized from to afford, after filtering and drying, 5alpha-Hydroxy-6beta-[2-(1H-imidazol-4-yl)ethylamino]cholestan-3beta-ol, malonate as a pale-yellow crystalline solid, having the following analytical properties: analysis found: C, 69.09; H, 9.77; N, 5.95%. H2O, 1.53%. Calculated for C35H59N3O6-0.51H2O: C, 69.05; H, 9.70; N, 5.92%. H2O, 1.51%

### EXAMPLE 10

### 5alpha-Hydroxy-6beta-[2-(1H-imidazol-4-yl)ethylamino]cholestan-3beta-ol, fumarate

(Trans)-butenedioic acid (fumaric acid, Fluka; 1.16 g, 10 mmol) is added to a solution of 5alpha-Hydroxy-6beta-[2-(1H-imidazol-4-yl)ethylamino]cholestan-3beta-ol (5.14 g, 10 mmole) in ethanol (20 ml). The solution is heated to 90° C., treated with water (18 g) and filtered. Upon cooling the product crystallizes and is filtered dried to afford 5alpha-Hydroxy-6beta-[2-(1H-imidazol-4-yl)ethylamino]cholestan-3beta-ol, fumarate as a pale-yellow crystalline solid, having the following analytical properties: analysis found: C, 68.01; H, 9.58; N, 6.63%. H2O, 1.05%. Calculated for C36H59N3O6-0.36H2O: C, 67.98; H, 6.58; N, 6.61%. H2O, 1.02%.

### EXAMPLE 11

### 5alpha-Hydroxy-6beta-[2-(1H-imidazol-4-yl)ethylamino]cholestan-3beta-ol, sulfate

A solution of sulfuric acid (Fluka, 5 ml, 1 M) in ethanol is added to a solution of 5alpha-Hydroxy-6beta-[2-(1H-imidazol-4-yl)ethylamino]cholestan-3beta-ol (2.57 g, 5 mmole) in hot ethanol (100 ml). The solution is evaporated to dryness under reduced pressure and the resulting residue is re-crystallized from ethanol. The product is filtered off and re-crystallized from ethanol to afford, after filtering and drying, 5alpha-Hydroxy-6beta-[2-(1H-imidazol-4-yl)ethylamino]cholestan-3beta-ol, sulfate as a pale-yellow crystalline solid, having the following analytical properties: analysis found: C, 62.84; H, 9.39; N, 6.94%. H2O, 5.72%. Calculated for C32H57N3O6S-1.92H2O: C, 62.81; H, 9.32; N, 6.87%. H2O, 5.65%

### EXAMPLE 12

### 5alpha-Hydroxy-6beta-[2-(1H-imidazol-4-yl)ethylamino]cholestan-3beta-ol, tosylate

5alpha-Hydroxy-6beta-[2-(1H-imidazol-4-yl)ethylamino]cholestan-3beta-ol (5.4 g, 10 mmol) is added to a solution of 4Methylbenzenesulfonic acid (Tosylate, Fluka; g 10 mmol) in hot toluene (40 ml). The solution is evaporated to dryness under reduced pressure and the resulting residue is re-crystallized from ethanol-ethyl acetate. The product is filtered off and dried, to afford 5alpha-Hydroxy-6beta-[2-(1H-imidazol-4-yl)ethylamino]cholestan-3beta-ol, tosylate as a pale-yellow crystalline solid, having the following analytical properties: analysis found : C, 69.52; H, 9.39; N, 6.28%. H2O, 1.77%. Calculated for C39H63N3O5S-0.64H20: C, 69.49; H, 9.35; N, 6.24%. H2O, 1.71%

### EXAMPLE 13

### 5alpha-Hydroxy-6beta-[2-(1H-imidazol-4-yl)ethylamino]cholestan-3beta-ol, mesylate

A solution of methylsulfonic acid (mesylic acid, Fluka, 5 ml, 1 M) in ethanol is added to a solution of 5alpha-Hydroxy-6beta-[2-(1H-imidazol-4-yl)ethylamino]cholestan-3beta-ol (2.57 g, 5 mmole) in hot ethanol (100 ml). The solution is evaporated to dryness under reduced pressure and the resulting residue is re-crystallized from ethanol. The product is filtered off and re-crystallized from ethanol to afford, after filtering and drying, 5alpha-Hydroxy-6beta-[2-(1H-imidazol-4-yl)ethylamino]cholestan-3beta-ol, mesylate as a pale-yellow crystalline solid, having the following analytical properties: analysis found: C, 66.10; H, 9.89; N, 7.05%. H2O, 1.63%. Calculated for C34H59N3O5S-0.53H2O: C, 66.06; H, 9.84; N, 7.01%. H2O, 1.59%

### EXAMPLE 14

### 5alpha-Hydroxy-6beta-[2-(1H-imidazol-4-yl)ethylamino]cholestan-3beta-ol, acetate

Acetic acid (sigma, 0.6 g, 10 mmol) is added to a solution of 5alpha-Hydroxy-6beta-[2-(1H-imidazol-4-yl)ethylamino]cholestan-3beta-ol (5.14 g, 10 mmole) in ethanol (40 ml). The solution is evaporated to dryness under reduced pressure and the resulting residue is re-crystallized from methanol. The product is filtered off and re-crystallized from ethanol to afford, after filtering and drying, 5alpha-Hydroxy-6beta-[2-(1H-imidazol-4-yl)ethylamino]cholestan-3beta-ol, acetate as a pale-yellow crystalline solid, having the following analytical properties: analysis found: C, 71.52; H, 10.38; N, 7.41%. H2O, 0.44%. Calculated for C34H59N3O4-0.13H2O: C, 71.50; H, 10.34; N, 7.36%. H2O, 0.41%

### EXAMPLE 15

### 5alpha-Hydroxy-6beta-[2-(1H-imidazol-4-yl)ethylamino]cholestan-3beta-ol, citrate

A mixture of 5alpha-Hydroxy-6beta-[2-(1H-imidazol-4-yl)ethylamino]cholestan-3beta-ol (5.4 g, 10 mmol) and 2-hydroxypropane-1,2,3-tricarboxylic acid (Fluka; 1.92 g 10 mmol) is heated in ethanol (40 ml). Water is added (25 ml). Upon cooling the product crystallizes and is filtered, dried to afford 5alpha-Hydroxy-6beta-[2-(1H-imidazol-4-yl)ethylamino]cholestan-3beta-ol, citrate as a pale-yellow crystalline solid, having the following analytical properties: analysis found: C, 66.06; H, 9.19; N, 6.12%. H2O, 2.11%. Calculated for C38H63N3O9-0.78H2O: C, 66.01; H, 9.12; N, 6.08%. H2O, 2.03%

### EXAMPLE 16

### 5alpha-Hydroxy-6beta-[2-(1H-imidazol-4-yl)ethylamino]cholestan-3beta-ol, L-Lactate

A mixture of 5alpha-Hydroxy-6beta-[2-(1H-imidazol-4-yl)ethylamino]cholestan-3beta-ol (5.4 g, 10 mmol) and 2(S)-hydroxypropanoic acid (L-lactate, Fluka; 3.88 g 10 mmol) is heated in ethanol (40 ml). The solution is evaporated to dryness under reduced pressure and the resulting residue is re-crystallized from acetone-ethanol. The product is filtered off and re-crystallized from ethyl acetate to afford, after filtering and drying, 5alpha-Hydroxy-6beta-[2-(1H-imidazol-4-yl)ethylamino]cholestan-3beta-ol, lactate as a pale-yellow crystalline solid, having the following analytical properties: analysis found: C, 70.53; H, 10.01; N, 6.88%. H2O, 0.43%. Calculated for C36H61N3O5-0.14H20: C, 70.50; H, 9.96; N, 6.85%. H2O, 0.41%

### EXAMPLE 17

### 5alpha-Hydroxy-6beta-[2-(1H-imidazol-4-yl)ethylamino]cholestan-3beta-ol, (D) (−) tartrate

5alpha-Hydroxy-6beta-[2-(1H-imidazol-4-yl)ethylamino]cholestan-3beta-ol (5.4 g, 10 mmol) is added to a solution of (2S,3S)2,3-dihydroxybutanedioic acid (D-(−)-tartaric acid; Fluka; 1.5 g 10 mmol) in ethanol (40 ml). The solution is evaporated to dryness under reduced pressure and the resulting residue is re-crystallized from ethanol. The product is filtered off and re-crystallized from to afford, after filtering and drying, 5alpha-Hydroxy-6beta-[2-(1H-imidazol-4-yl)ethylamino]cholestan-3beta-ol, -(D)-tartrate as a pale-yellow crystalline solid, having the following analytical properties: analysis found: C, 67.98; H, 9.48; N, 6.63%. H2O, 4.29%. Calculated for C36H61N3O8-1.51H2O: C, 67.94; H, 9.59; N, 6.61%. H2O, 4.27%.

### EXAMPLE 18

### Water Solubility

Solubility in water at room temperature of the acid addition salts of 5alpha-Hydroxy-6beta-[2-(1H-imidazol-4-yl)ethylamino]cholestan-3beta-ol of examples 1-17 was measured according to the following method:

An adequate amount of each sample was taken. Water (50% v/v) was added to each sample, and the mixture was shaken at room temperature for 5 hours. The supernatant was filtered through a filter, and diluted with a mixed solution of water 0.1% TFA/acetonitrile as necessary to give a sample solution. The concentration (mg/ml) of the sample solution was measured by high performance liquid chromatography (HPLC) with a calibration curve method, and taken as the solubility in water at room temperature.

HPLC Analysis Conditions:

HPLC purifications and analyses were carried out using an LC200 series Perkin Elmer apparatus, and diode array UV detector, using an Ultrasep C18 RP 100 column from Bischoff Chromatography.

HPLC analysis was done using an acetonitrile gradient (40% B for 8 min, then to 100% B in 20 min; A was 95:5 water/acetonitrile, 0.1% TFA; B was 95:5 acetonitrile/water 0.1% TFA) with a retention time of 18.5 min. Flow rate was 1 ml/min.

The results are listed in table 1. For comparison, the solubility of the free base is 0.47 mg/ml.

TABLE 1

| | Solubility in water at room temperature |
|---|---|
| Example | (mg/ml) |
| 1 | 23 |
| 2 | 42 |
| 3 | 46 |
| 4 | 28 |
| 5 | 31 |
| 6 | 31.5 |
| 7 | 1.4 |
| 8 | 11.2 |
| 9 | 55 |
| 10 | 16 |
| 11 | 18 |
| 12 | 24 |
| 13 | 11 |
| 14 | 4.5 |
| 15 | 7.9 |
| 16 | 65 |
| 17 | 37 |

1. An acid addition salt of 5α-hydroxy-6β-[2-(1H-imidazol-4-yl)ethylamino]cholestan-3β-ol formed with an acid selected from the group consisting of:
An inorganic acid,
An acyclic aliphatic carboxylic or sulfonic acid comprising no more than 8 carbon atoms, and
An aromatic carboxylic or sulfonic acid comprising no more than 4 aryl group.

2. The acid addition salt of 5α-hydroxy-6β-[2-(1H-imidazol-4-yl)ethylamino]cholestan-3β-ol according to claim 1, wherein said acid addition salt is formed with an inorganic acid selected from the group consisting of hydrochloride acid and sulfuric acid.

3. The acid addition salt of 5α-hydroxy-6β-[2-(1H-imidazol-4-yl)ethylamino]cholestan-3β-ol according to claim 1, wherein said acid addition salt is formed with an acyclic aliphatic carboxylic acid comprising between 2 and 6 carbon atoms, preferably between 3 and 4 carbon atoms.

4. The acid addition salt of 5α-hydroxy-6β-[2-(1H-imidazol-4-yl)ethylamino]cholestan-3β-ol according to claim 3, wherein said acyclic aliphatic carboxylic acid comprising between 2 and 6 carbon atoms, preferably between 3 and 4 carbon atoms, is a mono or dicarboxylic acid.

5. The acid addition salt of 5α-hydroxy-6β-[2-(1H-imidazol-4-yl)ethylamino]cholestan-3β-ol according to claim 3, wherein said acid addition salt is formed with succinic acid, glutaric acid, malonic acid, fumaric acid, or acetic acid.

6. The acid addition salt of 5α-hydroxy-6β-[2-(1H-imidazol-4-yl)ethylamino]cholestan-3β-ol according to claim 3, wherein said acyclic aliphatic carboxylic acid comprising between 2 and 6 carbon atoms, preferably between 3 and 4 carbon atoms, is substituted with at least one —OH.

7. The acid addition salt of 5α-hydroxy-6β-[2-(1H-imidazol-4-yl)ethylamino]cholestan-3β-ol according to claim 6, wherein said acid addition salt is formed with L-tartaric acid, D-tartaric acid, L-malic acid, citric acid, or 2(S)-hydroxypropanoic acid.

8. The acid addition salt of 5α-hydroxy-6β-[2-(1H-imidazol-4-yl)ethylamino]cholestan-3β-ol according to claim 1, wherein said acid addition salt is formed with an acyclic aliphatic sulfonic acid chosen from mesylic acid.

9. The acid addition salt of 5α-hydroxy-6β-[2-(1H-imidazol-4-yl)ethylamino]cholestan-3β-ol according to claim 1, wherein said acid addition salt is formed with an aromatic carboxylic acid comprising no more than 4 aryl groups, wherein at least one of said aryl group(s) is substituted with at least one —OH.

10. The acid addition salt of 5α-hydroxy-6β-[2-(1H-imidazol-4-yl)ethylamino]cholestan-3β-ol according to claim 9, wherein said acid addition salt is formed with 4,4′methylenebis[3-hydroxy-2-naphtoic acid.

11. The acid addition salt of 5α-hydroxy-6β-[2-(1H-imidazol-4-yl)ethylamino]cholestan-3β-ol according to claim 1, wherein said acid addition salt is formed with an aromatic carboxylic or sulfonic acid comprising no more than 3 aryl groups, preferably no more than one aryl group.

12. The acid addition salt of 5α-hydroxy-6β-[2-(1H-imidazol-4-yl)ethylamino]cholestan-3β-ol according to claim 11, wherein said acid addition salt is formed with benzenesulfonic acid, benzoic acid, or 4-methylbenzenesulfonic acid.

13. A pharmaceutical composition comprising a pharmaceutically acceptable carrier and the acid addition salt according to claim 1.

14. A method for treating neurodegenerative diseases, cancers or for activating the immune system of a patient, comprising administering to said patient an acid addition salt according to claim 1.

15. The acid addition salt of 5α-hydroxy-6β-2-(1H-imidazol-4-yl)ethylamino]cholestan-3β-ol according to claim 1, wherein said acid addition salt is formed with 2(S)-hydroxypropanoic acid, malonic acid, L-malic acid or tartric acid.
